(19) United States
(12) Patent Application Publication
(10) Pub. No.: US 2026/0033762 A1
(43) Pub. Date: Feb. 5, 2026

(54) Automated Urine Output System for Attachment to Hospital Bed

(71) Applicant: C. R. Bard, Inc., Franklin Lakes, NJ (US)

(72) Inventors: Eric A. Fallows, Apex, NC (US); Jill Walthall Jones, Avondale Estates, GA (US); Abigail A. Wilms, Tucker, GA (US); Kuilin Lai, Watkinsville, GA (US)

(21) Appl. No.: 19/356,931

(22) Filed: Oct. 13, 2025

Related U.S. Application Data
(63) Continuation of application No. 17/893,435, filed on Aug. 23, 2022, now Pat. No. 12,446,813.
(60) Provisional application No. 63/236,081, filed on Aug. 23, 2021.

(51) Int. Cl. A61B 5/20; A61F 5/44; A61B 5/00

(52) U.S. Cl. CPC A61B 5/208; A61F 5/44; A61B 5/6852

(57) ABSTRACT

A fluid monitoring system includes a fluid collection container, a monitoring module, and a console. The monitoring module can include a frame including a first frame member and a second frame member, the first frame member being displaceable with respect to the second frame member. The monitoring module can further include a coupling device configured to connect the fluid collection container to the frame, and a load cell operatively coupled between the first frame member and the second frame member. The load cell can be configured to produce an electrical signal in accordance with a weight of fluid accumulated in the fluid collection container. The console can include logic stored in memory that, when executed by one or more processors, causes performance of operations that include receiving the electrical signal from the load cell, converting the electrical signal into volumetric fluid data, and transmitting the volumetric fluid data.

### PRIORITY

This application is a continuation of U.S. Patent Application No. 17/893,435, filed August 23, 2022, now U.S. Patent No. 12,446,813, which claims the benefit of priority to U.S. Provisional Application No. 63/236,081, filed August 23, 2021, each of which is incorporated by reference in its entirety into this application.

### BACKGROUND

Today, nearly all physiological parameters of a patient admitted to a critical care unit are sensed automatically by commercial monitoring devices. This provides clinicians with invaluable information for interpreting the patient’s state. In most cases, these devices can also supervise whether the values of the physiological parameters they sense remain within a pre-established range set by the clinician. This range represents the values considered as acceptable for each parameter. If a parameter does not fall within its acceptable range, audible warnings to alert the health care staff are generated. These devices discharge the healthcare staff of a considerable workload, since the staff need not continuously supervise whether the physiological parameters of every patient lie within the acceptable range. They also avoid human errors, which are common in any repetitive task such as the supervision of physiological parameters.

It has been stated that the most relevant physiological parameter which is still measured and supervised manually by healthcare staff is urine output. Urine output is the best indicator of the state of the patient’s kidneys. If the kidneys are producing an adequate amount of urine, it means that they are well perfused and oxygenated. Otherwise, it is a sign that the patient is suffering from some complication. Urine output is required for calculating the patient’s water balance, which is essential in the treatment of burn patients. Finally, it is also used in multiple therapy protocols to check whether the patient reacts properly to treatment. When urine output is too low the patient is said to have oliguria. If the patient does not produce urine at all, then he/she is said to have anuria. Sometimes, urine output can be too high; in these cases, the patient is said to have polyuria.

It is common to measure urine output by collecting urine in a graduated container. Periodically the nursing staff manually records the reading of the container of every patient, and operates a valve which releases the urine into a larger container. As such, the healthcare staff does not benefit from the advantages of having an automated determination and recording of urine output, or of the continuous and automatic supervision of its values. In critical care units, measurements of most every patient’s urine output are often taken hourly, 24 times a day, 365 days a year. Furthermore, the monitoring of urine output for patients outside of the clinical setting is beneficial as well. As such urine collection devices that can be operated by the user/patient at home, for example, provide for urine output monitoring in the absence of a clinician. Transmitting the urine output data to the clinician provides for telemetric supervision of urine output information.

System and devices such as those described herein would decrease the workload associated with this collecting and monitoring urine output and, at the same time, permit supervision to take place on a more continuous basis resulting in better patient outcomes. Additionally, a compact system attached to the bed provides floor space for other medical equipment.

### SUMMARY OF THE INVENTION

Briefly summarized, disclosed herein is a system for monitoring urine output (UO) of a patient. The system includes a UO module configured to couple with a collection container. The UO module includes (i) a frame configured for anchoring to a bed, (ii) a load cell coupled with the frame, the load cell configured to define an electrical signal in accordance with a force defined by a weight of UO within the collection container, and (iii) a console including logic stored in memory that, when executed by one or more processors, causes performance of system operations. The operations include (i) receiving the electrical signal from the load cell, (ii) converting the electrical signal into volumetric UO data, and (iii) transmitting the UO data across a network to an external entity. The collection container is configured to collect UO therein and, in use the UO flows through a drainage tube extending between the patient and collection container.

In some embodiments, the system includes the collection container and the collection container may include a bag. The system may further include an identification device in the form of a barcode, a matrix code, or a radio-frequency identification (RFID) tag that is configured to couple with the collection container, and the identification device includes patient identification information.

The frame includes a first frame member and a second frame member coupled with the first frame member. The first frame member includes one or more hooks configured to anchor the first frame member to one or more frame rails of the bed. The second frame member includes a weight measurement arm configured to couple with the collection container, and in use, the collection container is hung from the weight measurement arm.

The first frame member may include a drainage tube retention device coupled thereto, where the drainage tube retention device is configured to secure a portion of the drainage tube to the first frame member to isolate movement of a proximal portion of the drainage tube from movement of a distal portion of the drainage tube. In some embodiments, the drainage tube retention device is configured to apply a clamping force to the drainage tube.

In some embodiments, the first frame member includes a first horizontal shelf disposed at a bottom of the first frame member, and the second frame member includes a second horizontal shelf disposed above the first horizontal shelf. The load cell may be coupled between the first horizontal shelf and the second horizontal shelf.

The system may further include an intermediate coupling device configured to couple the collection container with the weight measurement arm, and the intermediate coupling device may be configured to selectively couple with and decouple from the weight measurement arm. The intermediate coupling device may be further configured to couple with the collection container and inhibit decoupling of the intermediate coupling device from the collection container.

The intermediate coupling device may include an S-hook having an upper hook opening configured to receive the weight measurement arm and a lower hook opening configured to receive a coupling loop of the collection container. The S-hook may further include a latching arm rotatably coupled with the S-hook, where the latching arm (i) extends across the lower hook opening to define a closed position, (ii) is rotatable away from the closed position toward the lower hook opening to provide access to the lower hook opening, and (iii) is biased toward the closed position. In use, the coupling loop of the collection container may exert a force on the latching arm to rotate the latching arm away from the closed position allowing the lower hook opening to receive the coupling loop, and the latching arm may self-rotate back to the closed position inhibiting removal of the coupling loop from the lower hook opening. In use, the identification device may be attached to the S-hook.

In some embodiments, the operations may further include comparing the UO data with a UO limit stored in memory and as a result of the comparison, transmitting an alert to the external entity when the UO data exceeds the UO limit. The operations may further include obtaining identification information from the identification device and linking the UO data with the identification information.

Also disclosed herein is a method of monitoring urine output (UO) of a patient. The method includes coupling a UO collection container with a urinary catheter of the patient via a drainage tube extending between the catheter and the collection container and coupling the collection container with a UO monitoring system that includes a load cell configured to measure a weight of UO within the collection container. The method further includes (i) anchoring the UO monitoring system to a patient bed, (ii) establishing a flow of urine from the patient to the collection container, (iii) measuring a weight of UO within the collection container, (iv) converting weight of the UO into volumetric UO data, and (v) transmitting the UO data across a network to an external entity, where the external entity may include an electronic medical record.

The method may further include coupling an identification device with the collection container, where the identification device includes patient identification information, and the identification device is one of a barcode, a matrix code, or an RFID tag.

In some embodiments of the method, anchoring the UO monitoring system to a patient bed includes coupling one or more hooks of a first frame member of the UO monitoring system with one or more frame rails of the patient bed. A second frame member of the UO monitoring system may include a weight measurement arm, and the method may further include hanging the collection container from the weight measurement arm.

In some embodiments of the method, the first frame member further includes a drainage tube retention device configured to isolate movement of a distal portion of the tube from movement of a proximal portion of the tube, and the method further includes securing the drainage tube to the first frame member via the drainage tube retention device.

The method may further include coupling an intermediate coupling device of the UO monitoring system between the collection container and the weight measurement arm, where the intermediate coupling device is configured to couple with the collection container, and inhibit decoupling from the collection container. In some embodiments of the method, coupling an identification device with the collection container includes attaching the identification device to the intermediate coupling device.

The method may further include comparing the UO data with a UO limit stored in memory of the UO monitoring system, and as a result of the comparison, transmitting an alert to the external entity when the UO data exceeds the UO limit. The method may also further include obtaining identification information from the identification device, and linking the UO data with the identification information.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates an embodiment of a urine output monitoring system in use within a hospital environment, in accordance with some embodiments;

FIG. 2 illustrates a side view of the system of FIG. 1, in accordance with some embodiments;

FIG. 3 is an illustration of an intermediate coupling device of the system of FIG. 1, in accordance with some embodiments; and

FIG. 4 is a block diagram of a console of the system of FIG. 1, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

The directional terms “proximal” and “distal” are used herein to refer to opposite locations on a medical device. The proximal end of the device is defined as the end of the device closest to the end-user when the device is in use by the end-user. The distal end is the end opposite the proximal end, along the longitudinal direction of the device, or the end furthest from the end-user.

Unless defined otherwise, all technical and scientific terms used herein have the same meaning as commonly understood by those of ordinary skill in the art.

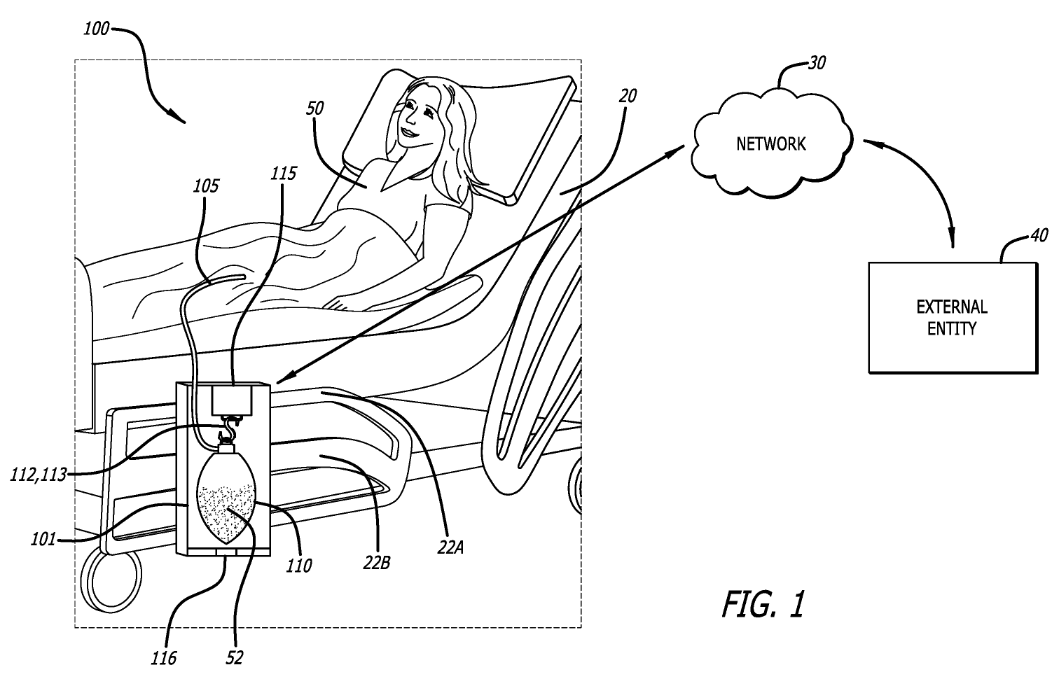

FIG. 1 illustrates an embodiment of a urine output (UO) monitoring system, in accordance with some embodiments disclosed herein. The UO monitoring system 100 is generally configured to automatically collect and monitor UO from a patient 50. The UO monitoring system 100 is further configured to automatically record UO data and share the UO data across a network 30 with an external entity 40. The UO monitoring system 100 generally includes a UO module 101 coupleable with a UO collection container 110 to measure a weight of the collection container 110 having urine 52 collected therein. In the illustrated embodiment, the UO monitoring system 100 includes an intermediate coupling device 112 coupled between the collection container 110 and the UO module 101 and the intermediate coupling device 112 may include an identification device 113 attached thereto.

The UO module 101 is selectively attachable to a bed 20, such as via bed frame rails 22A, 22B, for example. Attaching the UO module 101 to the bed 20 provides for co-location of the UO monitoring system 100 with the bed 20 which may be beneficial when the patient 50 along with the bed 20 is moved, such as between rooms, for example.

In use, the collection container 110 is fluidly coupled with a bladder of the patient 50 via a drainage tube 105 so that urine 52 flows through the drainage tube 105 to the collection container 110. The UO module 101 includes a console 115 coupled with a load cell (i.e., a force sensor) 116. The console 115 and the load cell 116 provide for automatically measuring and recording of a volume of urine 52 within the collection container 110 as further described below.

In use, the clinician may catheterize the patient 50 and connect a urinary catheter (not shown) to the drainage tube 105. The clinician may couple the collection container 110 with the UO module 101. The UO module 101 may automatically measure and record the volume of urine 52 within the collection container 110 to define UO data for the patient 50. The UO module 101 may transmit the UO data to the external entity 40 across the network 30 so that the clinician may view the UO data on a display of the external entity 40.

The network 30 represents the communication pathways between the UO module 101 and the external entity 40. In one embodiment, the network 30 is the Internet. The network 30 can also utilize dedicated or private communication links (e.g., WAN, MAN, or LAN) that are not necessarily part of the Internet. The network 30 may use standard communications technologies and/or protocols.

The external entity 40 may be a person, an institution, or a cloud computing environment (e.g., cloud computing resources accessible via a network such as the internet). In some embodiments, the external entity 40 may include a healthcare provider. As such, it may be possible for the clinician or other healthcare professional to access UO information of the UO monitoring system 100 and thereby remotely monitor the UO condition of the patient 50. In some embodiments, the UO monitoring system 100 may be configured to alert the external entity 40 of an extreme UO condition or trend.

In some embodiments, the external entity 40 may include access to an electronic medical record (EMR) of the patient 50. In such embodiments, the EMR may automatically record UO information for review by the healthcare provider. In further embodiments, the external entity 40 may include a user interface (not shown) to provide for manual entry of information such as an identification of the patient 50, for example.

Those of skill in the art will appreciate that the UO monitoring system 100 may contain other architectural modules that are not described herein. In addition, conventional elements, such as firewalls, authentication systems, payment processing systems, network management tools, load balancers, and so forth are not shown as they are not material to the invention.

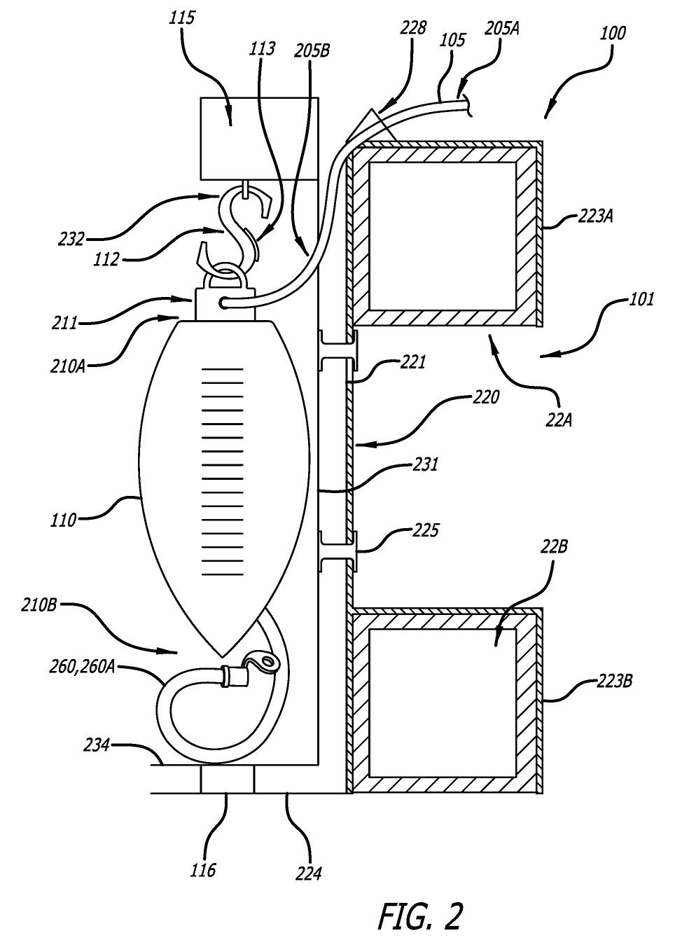

FIG. 2 is a side view illustration of the UO monitoring system 100, according to some embodiments. In some embodiments, the collection container 110 may be a separate component for use with the UO monitoring system 100. In other embodiments, the UO monitoring system 100 may include the collection container 110. The collection container 110 may be a flexible bag. As shown, the drainage tube 105 is coupled with the collection container 110 at a top end 210A of the collection container 110. An outlet port 260 is coupled with the collection container 110 at a bottom end 210B of the collection container 110. In some embodiments, the outlet port 260 may include a tube 260A extending away from the collection container 110. The collection container 110 includes an attachment component 211 at the top end 210A, and the attachment component 211 may include a loop portion of the collection container 110.

The collection container 110 may define a volume capacity to allow urine 52 to be collected and measured over an extend period of time (e.g., about 1–2 days) without draining the urine 52 from the collection container 110. In some embodiments, the collection container 110 may define a volume capacity exceeding about 1 liter, 2 liters, 3 liters or more.

With further reference to FIG. 2, the UO module 101 includes a frame 220 having a first frame member 221 coupleable with the frame rails 22A, 22B and a second frame member 231 coupleable with the collection container 110. The first frame member 221 includes frame hooks 223A, 223B configured to anchor the first frame member 221 to the frame rails 22A, 22B, respectively. In some embodiments, the first frame member 221 may include only one frame hook, such as the frame hook 223A, for example. Those of skill in the art will appreciate that the UO module 101 may be coupled with any structural members of the bed via clamps, adhesives, threaded fasteners, magnetic components, or any other suitable anchoring mechanism.

The first frame member 221 is coupled with the second frame member 231 via one or more resilient coupling components 225. The resilient coupling components 225 may constrain horizontal displacement of the second frame member 231 with respect to the first frame member 221 while allowing for vertical displacement of the second frame member 231 with respect to the first frame member 221.

The second frame member 231 includes a weight measurement arm 232 extending above the collection container 110. The weight measurement arm 232 is configured to couple with and suspend the collection container 110, i.e., in use, the collection container 110 hangs from the weight measurement arm 232.

In some embodiments, the second frame member 231 may include a bottom shelf 234 disposed horizontally beneath the collection container 110. In use, the outlet port 260 and/or a bottom portion of the collection container 110 may contact the bottom shelf 234 such that a portion of the weight of the collection container 110 may be supported by the bottom shelf 234. In some embodiments, the first frame member 221 may also include a bottom shelf 224 disposed beneath the bottom shelf 234 of the second frame member 231.

The load cell 116 is coupled between the first frame member 221 and the second frame member 231 so as to measure a vertically oriented force between the first frame member 221 and the second frame member 231. As the collection container 110 is coupled with the second frame member 231, the vertically oriented force between the second frame member 231 and the first frame member 221 is related to the weight of the collection container 110 including the urine 52 disposed therein. In the illustrated embodiment, the load cell 116 is coupled between the bottom shelf 224 of the first frame member 221 and the bottom shelf 234 of the second frame member 231. In other embodiments, the load cell 116 may be coupled between the first frame member 221 and the second frame member 231 at a location other than between the bottom shelf 224 of the first frame member 221 and the bottom shelf 234 of the second frame member 231.

The first frame member 221 includes a tubing retention device 228 attached thereto. The tubing retention device 228 is configured to secure the drainage tube 105 to the first frame member 221 so that movement of a distal portion 205A of the drainage tube 105 is isolated from movement of a proximal portion 205B of the drainage tube 105. In use, the patient 50 is coupled with the distal portion 205A of the drainage tube 105. As such, in some instances, movement of the patient 50 may result in movement of the distal portion 205A. Similarly, the proximal portion 205B of the drainage tube 105 is coupled with the collection container 110, and movement of the proximal portion 205B may affect the weight measurement of the collection container 110. The tubing retention device 228 is configured to prevent movement of the distal portion 205A from causing the proximal portion 205B to move. In some embodiments, the tubing retention device 228 may include two or more tubing engagement members (not shown) configured to contact the drainage tube 105 on opposites sides of the drainage tube 105. The tubing engagement members may define a clamping force on the circumference of the drainage tube 105 sufficient to prevent rotation and/or longitudinal displacement of the drainage tube 105 with respect to the tubing retention device 228.

The intermediate coupling device 112 (i.e., the S-hook) couples the weight measurement arm 232 of the UO module 101 with the attachment component 211 of the collection container 110. More specifically, in use, the intermediate coupling device 112 hangs on the weight measurement arm 232 and the collection container 110 hangs on the intermediate coupling device 112.

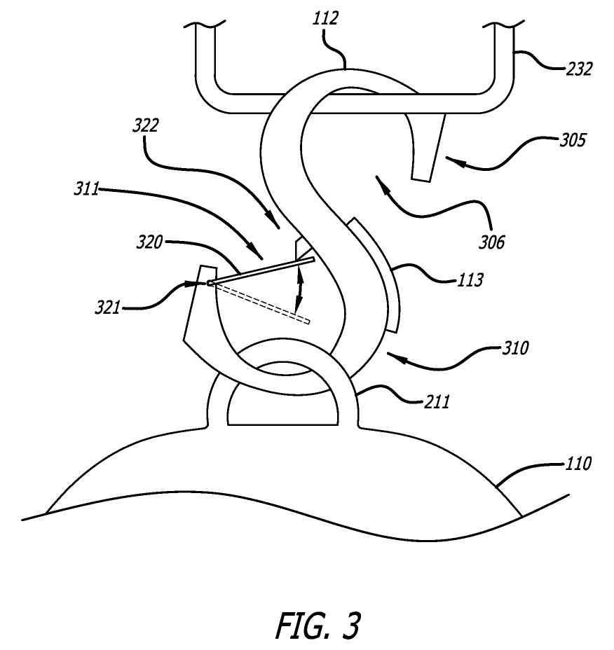

FIG. 3 is a detailed illustration of the intermediate coupling device 112, in accordance with some embodiments. The intermediate coupling device 112 generally includes (i) an upper coupling portion 305 for selective coupling with the weight measurement arm 232 and (ii) a lower coupling portion 310 for coupling with the attachment component 211 of the collection container 110.

The upper coupling portion 305 includes an upper opening 306 configured to receive the weight measurement arm 232 therein. The upper coupling portion 305 is configured to extend over the weight measurement arm 232 so that the weight of the collection container 110 is supported by the weight measurement arm 232. In some instances, the patient 50 may need to move away from the bed 20 (FIG. 1), and in such instances, the collection container 110 may need to be separated from the UO module 101. As such, the upper coupling portion 305 is configured for selective coupling to and decoupling from the weight measurement arm 232.

The lower coupling portion 310 is configured to couple with the collection container 110. However, in contrast to the upper coupling portion 305, the lower coupling portion 310 is configured to inhibit decoupling of the intermediate coupling device 112 from the collection container 110. More specifically, the lower coupling portion 310 is configured for extension through the loop of attachment component 211, and for retention within the loop of the attachment component 211.

In an exemplary embodiment, the intermediate coupling device 112 includes a latching arm 320 extending across an opening 311 of the lower coupling portion 310 to form a one-way hinge. The latching arm 320 is rotatable (clockwise as illustrated) about a pivot point 321 inward of the opening 311 to allow the lower coupling portion 310 to be inserted through the attachment component 211. The latching arm 320 is biased in the counterclockwise direction toward a mechanical stop 322 to define a closed position. The mechanical stop 322 prevents counterclockwise rotation of the latching arm 320 away from the closed position to inhibit extraction of the lower coupling portion 310 from the attachment component 211. As such, in use, the intermediate coupling device 112 may attach to the collection container 110 and may inhibit/prevent detachment from the collection container 110. As may be appreciated by one of ordinary skill, other suitable mechanisms could be employed to (i) allow connection of the intermediate coupling device 112 to the collection container 110 and (ii) prevent disconnection of the intermediate coupling device 112 from the collection container 110, and such other suitable mechanisms are therefore disclosed herein.

As stated above, the UO monitoring system 100 further includes an identification device 113 coupled with the intermediate coupling device 112. The identification device 113 may be a barcode, a matrix code (e.g., a quick response (QR) code) or a radio-frequency identification (RFID) tag. The identification device 113 may include patient specific information. In use, the identification device 113 may be attached to the intermediate coupling device 112 by the clinician. In some embodiments, the identification device 113 may include human readable information. Attaching of the intermediate coupling device 112 together with the identification device 113 to the collection container 110, links the collection container 110, the urine 52 collected within the collection container 110, and the UO data to the patient 50.

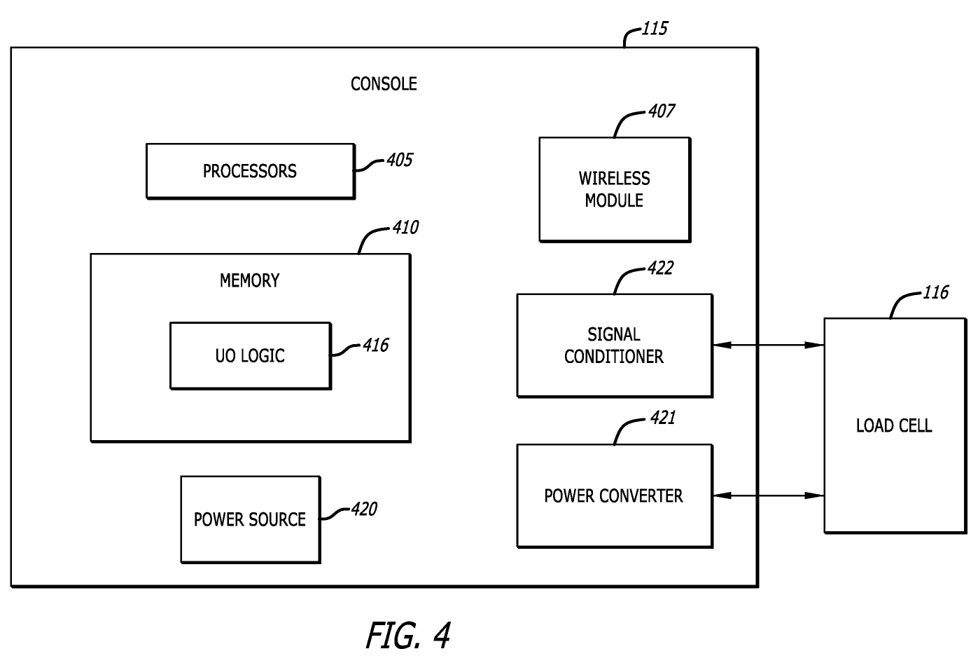

FIG. 4 illustrates a block diagram of the console 115 of FIG. 1, in accordance with some embodiments. The console 115 includes one or more processors 405 and memory 410 including a non-transitory, computer-readable storage medium. Stored in the memory 410 is UO logic 416. A power source 420 provides electrical power to the console 115 including the console components and the load cell 116. The load cell 116 is coupled with the console 115. A signal conditioner 422 converts electrical signals from the load cell 116 to digital signals for processing by the UO logic 416. The electrical power passes through a power converter 421 to facilitate the operation of the load cell 116. A wireless module 407 facilitates communication and data sharing with the external entity 40.

The power source 420 may be an external facility power source. The power source 420 may also include a rechargeable battery configured to provide power when the UO module 101 is disconnected from the power source 420. The power converter 421 converts the electrical power from the power source 420 into forms of electrical power compatible with the load cell 16 and the console components.

The UO logic 416 is configured to control the load cell 116 and process electrical signals received from the load cell 116. The UO logic 416 may also control the power converter 421 to enable/disable the load cell 116. The UO logic 416 receives digital signals from the load cell 116 via the signal conditioner 422 and processes the digital signals into UO data. More specifically, the UO logic 416 converts the digital signals from the load cell 116 into a weight of the urine 52 within the collection container 110 and then further converts the weight of the urine 52 into a volume of the urine 52. The UO logic 416 records and prepares the UO data for transfer to the external entity 40.

The UO logic 416 may receive identification information (e.g., the identification of the patient 50 included on the identification device 113) and link the UO data to the identification of the patient 50. In some embodiments, the UO logic 416 may receive the identification information via the wireless module 407, and in further embodiments, the wireless module 407 may be configured to receive the identification information from an RFID tag.

In some embodiments, the UO logic 416 may be configured to generate an alert (e.g., a warning) in the event of an extreme UO condition or trend. More specifically, the UO logic 416 may compare the UO data with one or more limits stored in memory 410. As a result of the comparison, the UO logic 416 may transmit the alert to the external entity 40. In one exemplary embodiment, the UO logic 416 may compare the UO data with a low UO limit and generate an alert when the UO data indicates a UO below the low UO limit.

In the illustrated embodiment, the UO logic 416 may be in the form of a software application that is loaded on the UO module 101 and executable by the one or more processors 405. In other embodiments, the UO logic 416 or portions thereof, need not be loaded on the UO module 101 but may instead execute within a cloud computing environment (which may also be represented by the reference numeral 30) such that UO data obtained by the UO module 101 are communicated to the UO logic 416 for processing. Thus, the UO logic 416 represented as being part of the UO module 101 may include an application programming interface (API) that is configured to transmit and receive data communication messages to and from the UO logic 416 operating in the cloud computing environment.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

What is claimed is:

1. A fluid monitoring system, comprising:
a fluid collection container;
a monitoring module, comprising:
a frame including a first frame member and a second frame member, wherein the first frame member is displaceable with respect to the second frame member;
a coupling device configured to connect the fluid collection container to the frame; and
a load cell operatively coupled between the first frame member and the second frame member, wherein the load cell is configured to produce an electrical signal in accordance with a weight of fluid accumulated in the fluid collection container; and
a console including logic stored in memory that, when executed by one or more processors, causes performance of operations that include receiving the electrical signal from the load cell, converting the electrical signal into volumetric fluid data, and transmitting the volumetric fluid data.

2. The fluid monitoring system according to claim 1, wherein transmitting the volumetric fluid data comprises transmitting across a network to an external entity.

3. The fluid monitoring system according to claim 1, wherein the coupling device is configured to suspend the fluid collection container from the frame.

4. The fluid monitoring system according to claim 1, wherein the frame is configured for anchoring to a bed.

5. The fluid monitoring system according to claim 1, further comprising a catheter assembly in fluid communication with the collection container.

6. The fluid monitoring system according to claim 5, further comprising a retention device coupled to the frame, the retention device configured for securement of the catheter assembly with respect to the frame.

7. The fluid monitoring system according to claim 1, wherein the fluid collection container comprises an identification device including patient identification information, wherein the identification device is one of a barcode, a matrix code, or a radio-frequency identification (RFID) tag.

8. The fluid monitoring system according to claim 7, wherein the identification device is included with the coupling device.

9. The fluid monitoring system according to claim 1, wherein the coupling device comprises a weight measurement arm.

10. The fluid monitoring system according to claim 9, wherein the coupling device is configured to inhibit disconnection of the fluid collection container.

11. The fluid monitoring system according to claim 10, wherein the coupling device comprises an S-hook including an upper hook configured to receive the weight measurement arm, and a lower hook configured to receive a coupling loop of the fluid collection container.

12. The fluid monitoring system according to claim 11, wherein the S-hook further comprises a rotatable latching arm.

13. The fluid monitoring system according to claim 12, wherein, in use:
the coupling loop of the collection container exerts a force on the rotatable latching arm to rotate the rotatable latching arm away from a closed position, allowing the lower hook to receive the coupling loop, and
the rotatable latching arm self-rotates back to the closed position, inhibiting removal of the coupling loop from the lower hook.

14. The fluid monitoring system according to claim 1, wherein the first frame member includes a first shelf, wherein the second frame member includes a second shelf, and wherein the load cell is positioned between the first shelf and the second shelf.

15. The fluid monitoring system according to claim 1, wherein the first frame member is coupled to the second frame member via one or more resilient coupling components.

16. The fluid monitoring system according to claim 15, wherein the one or more resilient coupling components are configured to constrain horizontal displacement of the second frame member with respect to the first frame member.

17. The fluid monitoring system according to claim 16, wherein the one or more resilient coupling components are configured to permit vertical displacement of the second frame member with respect to the first frame member.
